(19) United States
(12) Patent Application Publication
(10) Pub. No.: US 2022/0031715 A1
(43) Pub. Date: Feb. 3, 2022

(54) COMPOSITIONS AND METHODS FOR ENHANCING WOUND HEALING

(71) Applicant: Cocky Smart Pty Ltd, Crossman (AU)

(72) Inventor: John Terence Chamberlain, Crossman (AU)

(21) Appl. No.: 17/275,918

(22) PCT Filed: Sep. 12, 2019
(86) PCT No.: PCT/AU2019/050978

§ 371 Date: Mar. 12, 2021

(30) Foreign Application Priority Data
Sep. 14, 2018 (AU) 2018903467

(51) Int. Cl. A61K 31/661; A61P 17/02; A61K 9/00; A61L 15/20; A61L 15/44

(52) U.S. Cl. CPC A61K 31/661; A61P 17/02; A61L 2300/216; A61L 15/20; A61L 15/44; A61K 9/0014

(57) ABSTRACT

A method of enhancing wound healing in a subject, comprising administering to the wound a therapeutically effective amount of triphenyl phosphate (TPP) or a variant thereof.

### TECHNICAL FIELD

The present invention relates to methods and compositions that support and enhance wound healing.

### BACKGROUND ART

Wound healing is a complex process in which the skin, and the tissues under it, repair themselves after injury. In undamaged skin, the epidermis (surface layer) and dermis (deeper layer) form a protective barrier against the external environment. When the barrier is broken, a regulated sequence of biochemical events is set into motion to repair the damage. This process is divided into predictable phases: blood clotting (haemostasis), inflammation, new tissue growth (proliferation), and tissue remodelling (maturation). Blood clotting may be considered to be part of the inflammation stage instead of a separate stage.

During proliferation, angiogenesis, collagen deposition, granulation tissue formation, epithelialization, and wound contraction occur. In angiogenesis, vascular endothelial cells form new blood vessels. In fibroplasia and granulation tissue formation, fibroblasts grow and form a new, provisional extracellular matrix (ECM) by excreting collagen and fibronectin. Concurrently, re-epithelialization of the epidermis occurs, in which epithelial cells proliferate and ‘crawl’ atop the wound bed, providing cover for the new tissue. In wound contraction, myofibroblasts decrease the size of the wound by gripping the wound edges and contracting using a mechanism that resembles that in smooth muscle cells. When the cells' roles are close to complete, unneeded cells undergo apoptosis.

The wound healing process is not only complex but also fragile, and it is susceptible to interruption or failure leading to the formation of non-healing chronic wounds. Factors that contribute to non-healing chronic wounds are diabetes, venous or arterial disease, infection, and metabolic deficiencies of old age.

There is a need for methods and compositions to support and enhance wound healing; or at least the provision of methods and compositions to compliment the previously known wound healing treatments.

The present invention seeks to provide an improved or alternative method for enhancing wound healing.

The previous discussion of the background art is intended to facilitate an understanding of the present invention only. The discussion is not an acknowledgement or admission that any of the material referred to is or was part of the common general knowledge as at the priority date of the application.

### SUMMARY OF INVENTION

In a first aspect, the invention provides a composition comprising:

a) triphenyl phosphate (TPP) or variant thereof; and

b) a suitable carrier, excipient or diluent thereof.

Preferably, the composition enhances wound healing in a subject in need thereof. In one embodiment, the TPP variant is any tri-aryl phosphate which at least partially enhances wound healing in a subject in need thereof.

Preferably, the composition comprises a suitable carrier selected from the group consisting of: a pharmaceutically acceptable carrier and a carrier adapted for the administration to animals.

The composition may be adapted for topical administration to a subject in need thereof. The concentration of TPP in the composition is dependent on individual circumstances. For example, the concentration of TPP in the composition is selected from the group consisting of: between 10 g/l and 200 g/l and between 50 g/l and 150 g/l.

In a second aspect, the invention provides a pharmaceutical composition comprising:

a) triphenyl phosphate (TPP) or variant thereof; and

b) one or more pharmaceutically acceptable carriers, excipients or diluents thereof.

Preferably, the pharmaceutical composition is adapted for topical administration to a subject in need thereof.

The concentration of TPP in the composition is dependent on individual circumstances. For example, the concentration of TPP in the composition or the pharmaceutical composition is selected from the group consisting of: between 10 g/l and 200 g/l and between 50 g/l and 150 g/l.

The pharmaceutical composition may further comprise additional ingredients. For example, the pharmaceutical composition further comprises a hydrocarbon solvent. Preferably, the hydrocarbon solvent is petroleum naptha. In some embodiments, the concentration of hydrocarbon solvent is selected from the group consisting of: between 5% and 30% w/w and between 10% and 20% w/w.

In a further example, the pharmaceutical composition further comprises an alcohol solvent. Preferably, the alcohol solvent is ethanol. In some embodiments, the amount of alcohol solvent is selected from the group consisting of: between 1% and 20% w/w and between 5% and 10% w/w.

The pharmaceutical composition may be a liquid, ointment, spray, cream or gel.

In a third aspect, the invention provides a method of enhancing wound healing in a subject in need thereof, comprising administering to the wound of the subject, a therapeutically effective amount of the pharmaceutical composition.

In a particular aspect, the wound healing occurs at an increased rate compared with a wound to which the composition is not administered.

The wound may be selected from the group comprising: chronic wounds, ulcers, diabetic ulcers, burns, abrasions, lacerations, punctures and avulsions.

Preferably, the subject is human.

In another preferred embodiment, the composition is administered topically. The composition may be administered on a bandage which is them applied to the wound as a dressing. The composition may be directly administered to the wound.

The amount of TPP administered is dependent on individual circumstances. For example, the composition may be administered at between 2 mg and 100 mg of TPP per cm² of skin. Preferably, the composition is administered at between about 5 mg and 50 mg of TPP per cm² of skin.

The frequency of application will depend on the individual circumstances. For example, the composition is administered using a regimen selected from the group consisting of: hourly, three times daily, twice daily, once daily, once every two days, once every three days, once weekly, once every two weeks, once monthly, once every two months, once every six months, and once yearly. The composition may be administered using a regimen of administration on days 0, 1, 3, 6, 8, 10 and 13, where day 0 is the day on which the wound was sustained.

In a fourth aspect, the invention provides a wound dressing comprising the pharmaceutical composition.

In a fifth aspect, the invention provides a composition comprising triphenyl phosphate (TPP) for use in a method of enhancing wound healing.

In a sixth aspect, the invention provides the use of triphenyl phosphate (TPP) or variant thereof in the manufacture of a medicament for enhancing wound healing in a subject in need thereof.

In a seventh aspect, the invention provides a kit for enhancing wound healing in a subject comprising the pharmaceutical composition. Preferably, the kit comprises instructions for its use.

The present invention provides a topical composition for enhancing wound healing in a subject in need thereof, said composition comprising:
a) triphenyl phosphate or variant thereof.

The present invention provides a method of enhancing wound healing in a subject in need thereof, comprising the step of:
i) applying a topical composition comprising:
   a) triphenyl phosphate or variant thereof.
      to the wound.

The present invention provides a method for the manufacture of a topical composition for enhancing wound healing in a subject in need thereof, said composition comprising:
a) triphenyl phosphate or variant thereof.

The present invention provides for the use of a topical composition comprising:
a) triphenyl phosphate or variant thereof.
   for enhancing wound healing in a subject in need thereof.

The present invention provides a kit comprising
i) a topical composition for enhancing wound healing in a subject in need thereof, said composition comprising:
b) triphenyl phosphate or variant thereof and
c) instructions for use.

Preferably, the amount of triphenyl phosphate is between 10 g/l and 200 g/l, and more preferably 50 g/l or 150 g/l, or somewhere in between.

Preferably the composition further comprises a hydrocarbon solvent such as petroleum naphtha. Preferably, the amount of hydrocarbon solvent is between 5% and 30% w/w, and more preferably between 10% and 20% w/w.

Preferably the composition further comprises an alcohol solvent such as ethanol. Preferably, the amount of alcohol solvent is between 1% and 20% w/w, and more preferably between 5% and 10% w/w.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 is a diagram of wound placement on subject pigs.

FIG. 2 is a diagram of the wounds sampled for analysis. Sampling was undertaken on Days 6, 8, 10 and 13. Gang #1=Days 10 and 13. Gang #2=Days 6 and 8. Wounds were initially 2 cm in diameter and about 0.5 cm deep.

FIG. 3 is a graph of wound size over time of a typical animal for the entire time course of the experiment.

FIG. 4 is photographs of Day 13 wounds treated with either CS4652 or 0.9% saline Control.

FIG. 5 is a graph of the average wound size for all animals over the later stages (Days 8, 10 and 13) A: CS4652 solvent carrier, B: 150 g/L CS4652, C: 50 g/L CS4652, D: 0.9% Saline Control.

FIG. 6 is a plot of the gene products of the Matrix Metalloproteinase 2 gene during wound healing.

### DESCRIPTION OF INVENTION

### Detailed Description of the Invention

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

As used herein, the term “about” refers to a quantity, level, value, dimension, size, or amount that varies by as much as 30%, 25%, 20%, 15% or 10% to a reference quantity, level, value, dimension, size, or amount.

It is to be understood that, if any prior art publication is referred to herein, such reference does not constitute an admission that the publication forms a part of the common general knowledge in the art.

The term “aryl” means any functional group or substituent derived from an aromatic ring, including an aromatic carbocyclic or heterocyclic ring.

The term “carrier adapted for the administration to animals” means one or physiologically compatible materials that do not interfere with the effectiveness of the biological activity of the active ingredients in an animal, including solvents, dispersion media, coatings, antibacterial and antifungal agents, isotonic and absorption delaying agents, and the like.

The terms “enhance” or “enhanced” in respect of wound healing are used to convey that the present invention changes either the appearance, form, characteristics and/or the physical attributes of the tissue to which it is being provided, applied or administered compared to wounds that have not had the composition of the present invention applied. The change in form may be demonstrated by any of the following alone or in combination: more rapid wound repair; decreased inflammation of the skin; decreased redness; reduction of scarring; reduction in raw wound size; closure of wounds and lesions; a reduction in symptoms including, but not limited to, pain, inflammation, itching; or other symptoms associated with wound healing, inflammatory conditions or the like. In one aspect of the invention, the term “enhance” is used to describe a more rapid reduction in raw wound size compared to wounds that have not had the composition of the present invention applied. Measures of wound healing may be developed from references such as Sorg et al Skin Wound Healing: An Update on the Current Knowledge and Concepts Eur Surg Res 2017; 58:81-94.

The term “pharmaceutically acceptable carrier” means one or physiologically compatible materials that do not interfere with the effectiveness of the biological activity of the active ingredients, including solvents, dispersion media, coatings, antibacterial and antifungal agents, isotonic and absorption delaying agents, and the like.

The term “subject in need thereof” means any human or animal, including any mammal, an agricultural animal (such as a pig, cow, sheep, horse, goat etc.), domestic pet (such as a cat, dog etc.), or non-agricultural animal.

The term “therapeutically effective amount” means the amount necessary to bring about a therapeutic response in a subject. Preferably, the term “therapeutically effective amount” means the amount necessary to enhance wound healing.

As used herein, the terms “topically” and “topical administration” means directly applying, laying on or spreading on the body surface, such as on the outer skin.

The term “triphenyl phosphate” or “TPP” means a chemical compound with the formula OP(OC₆H5)₃, and PubChem ID 8289. The person skilled in the art will understand that there may be variants of TPP that can also be used in the present invention. The term “variants thereof” in respect of TPP means any tri-aryl phosphate which at least partially enhances wound healing in a subject in need thereof. Suitable methods to test and select a tri-aryl phosphate for use in the invention are described herein and well known to the person skilled in the art. For example, the person skilled in to the art may use in vitro cutaneous skin cell culture models and/or may use ex vivo or in vivo wound healing models to select suitable tri-aryl phosphates.

As used herein, the term “wound” refers to physical disruption of the continuity or integrity of tissue structure. Wounds included may be acute or chronic and include cuts and lacerations, surgical incisions or wounds, punctures, grazes, scratches, compression wounds, abrasions, friction wounds, decubitus ulcers (e.g. pressure or bed sores); thermal effect wounds (burns from old and heat sources), chemical wounds (e.g. acid or alkali burns) or pathogenic infections (e.g. viral, bacterial or fungal) including open or intact boils, skin eruptions, blemishes and acne, ulcers, chronic wounds, (including diabetic-associated wounds such as lower leg and foot ulcers, venous leg ulcers and pressure sores), skin graft/transplant donor and recipient sites, immune response conditions, e.g. psoriasis and eczema, stomach or intestinal ulcers, oral wounds, including a ulcers of the mouth, damaged cartilage or bone, amputation wounds and corneal lesions.

### Composition

The present invention provides a composition comprising triphenyl phosphate and a carrier. In some embodiments, the present invention provides a topical composition for enhancing wound healing in a subject in need thereof, said composition comprising:
a) triphenyl phosphate or any variant thereof.

Triphenyl Phosphate (TPP—PubChem CID8289) is a compound commonly used in fire retardation applications and as a plasticizer in nail polish and other cellulose lacquers. It has been demonstrated to have very low toxicity in mammals. To our knowledge, the effect of TPP on wound healing has not been assessed.

In one example, the TPP enhances wound healing activity through the activation or inhibition of hormone receptors that have been implicated in wound healing. It has been reported that TPP activates at least six hormone receptors that enhance wound healing (NR1I2/PAR, NR1i2/PXR, ESR1, NFE2L2/HEBP1, NR1 L3/CAR, and TSHR). It has further been reported that TPP inhibits at least one receptor, the human androgen receptor alpha (AR/HUMARA), which has been shown to impair wound healing and has been implicated in the loss of wound healing efficacy with age.

In specific embodiments, the amount of triphenyl phosphate (TPP) in the composition is selected from the following group: at least 2 g/l, 3 g/l, 4 g/l, 5 g/l, 6 g/l, 7 g/l, 8 g/l, 9 g/l, 10 g/l, 11 g/l, 12 g/l, 13 g/l, 14 g/l, 15 g/l, 20 g/l, 25 g/l, 30 g/l, 35 g/l, 40 g/l, 45 g/l, 50 g/l, 55 g/l, 60 g/l, 65 g/l, 70 g/l, 75 g/l, 80 g/l, 85 g/l, 90 g/l, 95 g/l and 97 g/l. In specific embodiments, the maximum concentration of the TPP is 50 g/l, 100 g/l, 150 g/l, 200 g/l, 250 g/l or 300 g/l. The relative amount of TPP may be between 1 g/l and 300 g/l, 5 g/l and 250 g/l, 5 g/l and 220 g/l, 10 g/l and 250 g/l, 10 g/l and 200 g/l. Preferably the amount of triphenyl phosphate is 50 g/l or 150 g/l, or somewhere in between.

The composition may further comprise any suitable carrier. Preferably, the carrier is a pharmaceutically acceptable carrier or a carrier that is adapted for the administration to animals.

Generally, examples of suitable carriers, excipients and diluents include, without limitation, water, saline, ethanol, dextrose, glycerol, lactose, dextrose, sucrose, sorbitol, mannitol, starches, gum acacia, calcium phosphates, alginate, tragacanth, gelatine, calcium silicate, microcrystalline cellulose, polyvinylpyrrolidone, cellulose, water syrup, methyl cellulose, methyl and propylhydroxybenzoates, polysorbates, talc magnesium stearate, mineral oil or combinations thereof. The compositions can additionally include lubricating agents, pH buffering agents, wetting agents, emulsifying and suspending agents, preserving agents, sweetening agents or flavouring agents.

The composition may further comprise a hydrocarbon solvent such as petroleum naphtha, preferably petroleum naphtha 150 (e.g. Recosol 150) or other grade of petroleum naphtha. The hydrocarbon solvent should be chosen such that it can dissolve TPP to at least the extent necessary to provide the amount of active in the topical composition as claimed and optimises the viscosity of the formulation, or at least does not interfere with the ability of the alcohol solvent and/or solvent oil to dissolve the TPP. The hydrocarbon solvent chosen should be pharmaceutically acceptable, preferably a hydrocarbon solvent that is generally recognized as safe (GRAS). Any hydrocarbon mixture comprising predominantly of straight-chained and cyclic aliphatic hydrocarbons having at least seven carbons per molecule, more preferably from seven to nine carbon atoms per molecule. The hydrocarbon mixture preferably has a boiling point between 90° C. and 200° C. By predominantly, it is preferred that the hydrocarbon mixture has at least 51%, 60%, 70%, 80%, 90%, 95% or 99% of straight-chained and cyclic aliphatic hydrocarbons having at least seven carbons per molecule.

It has been also found by the present inventors that the presence of petroleum naphtha may also increase the wound healing enhancing abilities of the compositions of the present invention. Thus the hydrocarbon may serve the dual roles of solvent and active agent to enhance wound healing.

In specific embodiments, the relative amount of hydrocarbon solvent in the composition is selected from the following group consisting of: at least 2% w/w, 3% w/w, 4% w/w, 5% w/w, 6% w/w, 7% w/w, 8% w/w, 9% w/w, 10% w/w, 11% w/w, 12% w/w, 13% w/w, 14% w/w, 15% w/w, 20% w/w, 25% w/w, 30% w/w, 35% w/w, 40% w/w, 45% w/w, 50% w/w, 55% w/w, 60% w/w, 65% w/w, 70% w/w, 75% w/w, 80% w/w, 85% w/w, 90% w/w, 95% w/w and 97% w/w. In specific embodiments, the maximum concentration of the hydrocarbon solvent is 50% w/w, 60% w/w, 70% w/w, 80% w/w, 90% w/w, 95% w/w or 97% w/w. The relative amount of hydrocarbon solvent in the composition may be between 1% w/w and 97% w/w, 10% w/w and 97%, 10% w/w and 90% w/w, 5% w/w and 50% w/w, 5% w/w and 30% w/w. Preferably, the amount of hydrocarbon solvent is between 10% and 20% or about 13.5% w/w.

The composition may further comprise an alcohol solvent. The alcohol solvent is preferably chosen such that it dissolves TPP to at least the extent necessary to provide the amount of active in the topical composition as claimed and optimises the viscosity of the formulation. Advantageously, in some embodiments, the alcohol solvent is selected from the group consisting of: 02-6 alcohols or C2-4 alcohols, and combinations thereof. The alcohol solvent may, for example, be a low molecular weight alcohol such as methanol, isopropyl alcohol (isopropanol), propanol, 2-butanol, n-butanol and ethyl alcohol (or ethanol), and combinations thereof. Other alcohol solvents will be clear to the skilled reader. Preferably the alcohol solvent is ethanol. The alcohol solvent chosen should be pharmaceutically acceptable, preferably an alcohol solvent that is generally recognized as safe (GRAS).

In specific embodiments, the relative amount of alcohol solvent in the composition is selected from the following group consisting of: at least 2% w/w, 3% w/w, 4% w/w, 5% w/w, 6% w/w, 7% w/w, 8% w/w, 9% w/w, 10% w/w, 11% w/w, 12% w/w, 13% w/w, 14% w/w, 15% w/w, 20% w/w, 25% w/w, 30% w/w, 35% w/w, 40% w/w, 45% w/w, 50% w/w, 55% w/w, 60% w/w, 65% w/w, 70% w/w, 75% w/w, 80% w/w, 85% w/w, 90% w/w, 95% w/w and 97% w/w. In specific embodiments, the maximum concentration of the alcohol solvent in the composition is 50% w/w, 60% w/w, 70% w/w, 80% w/w, 90% w/w, 95% w/w or 97% w/w. The relative amount of alcohol solvent may be between 1% w/w and 97% w/w, 1% w/w and 90%, 1% w/w and 70% w/w, 1% w/w and 30% w/w, 1% w/w and 20% w/w. Preferably, the amount of alcohol solvent in the composition is between 5% and 10% w/w or about 8%.

The composition may further comprise a solvent oil that is a glyceride, preferably a triglyceride. However, mono and diglyceride solvent oils are also contemplated. The solvent oil may act as a solvent for the TPP in the composition of the present invention. The solvent oil is preferably chosen such that it dissolves TPP to at least the extent necessary to provide the amount of active in the topical composition as claimed, or at least does not interfere with the ability of the hydrocarbon solvent and/or alcohol solvent to dissolve the TPP. Alternatively, the TPP may dissolve in a mixture of the hydrocarbon solvent and/or alcohol solvent and/or solvent oil. Preferably the oil may be chosen from linseed oil and pine oil. Both of these oils have anti-inflammatory properties, and may assist in enhancing wound healing. Other oils that may be used include olive oil, coconut oil, eucalyptus oil, tea tree oil, clove oil, rose oil, thyme oil, castor oil, sesame oil. The solvent oil chosen should be pharmaceutically acceptable, and preferably a solvent oil that is generally recognized as safe (GRAS).

In specific embodiments, the relative amount of solvent oil in the composition is selected from the following group consisting of: at least 2% w/w, 3% w/w, 4% w/w, 5% w/w, 6% w/w, 7% w/w, 8% w/w, 9% w/w, 10% w/w, 11′)/ow/w, 12% w/w, 13% w/w, 14% w/w, 15% w/w, 20% w/w, 25% w/w, 30% w/w, 35% w/w, 40% w/w, 45% w/w, 50% w/w, 55% w/w, 60% w/w, 65% w/w, 70% w/w, 75% w/w, 80% w/w, 85% w/w, 90% w/w, 95% w/w and 97% w/w. In specific embodiments, the maximum concentration of the solvent oil is 50% w/w, 60% w/w, 70% w/w, 80% w/w, 90% w/w, 95% w/w or 97% w/w. The relative amount of solvent oil may be between 1′)/ow/w and 97% w/w, 10% w/w and 97%, 10% w/w and 90% w/w, 50% w/w and 97% w/w, 50% w/w and 95% w/w.

In one form of the invention, the composition may therefore comprise one or more of the following:
a) petroleum naphtha;
b) pine oil;
c) linseed oil;
d) ethanol; and/or
e) isopropyl alcohol

Preferably, the composition comprises:
i) between 10 g/l and 200 g/l TPP;
ii) between 5% and 30% w/w hydrocarbon solvent; and/or
iii) between 1% and 20% w/w alcohol solvent.

More preferably, the composition comprises:
i) between 10 g/l and 200 g/l TPP;
ii) between 5% and 30% w/w petroleum naphtha; and/or
iii) between 1% and 20% w/w ethanol.

More preferably, the composition comprises:
i) between 50 g/l and 150 g/l TPP;
ii) between 10% and 20% w/w petroleum naphtha; and/or
iii) between 5% and 10% w/w ethanol.

Method of Treatment

The present invention provides a method of enhancing wound healing, comprising the step of:
iv) applying a topical composition comprising:
   a) triphenyl phosphate or a variant thereof to the wound.

The present invention provides for the use of a topical composition comprising:
a) triphenyl phosphate or a variant thereof
   for enhancing wound healing in a subject in need thereof.

In another embodiment, the present invention provides a method of enhancing would healing in a subject in need thereof, comprising administering to the wound of the subject, a therapeutically effective amount of a pharmaceutical composition comprising triphenyl phosphate.

The present invention provides a method for the manufacture of a topical composition for enhancing wound healing in a subject in need thereof, said composition comprising:
a) triphenyl phosphate or a variant thereof.

Preferably, the amount of triphenyl phosphate is between 10 and 200 g/l. More preferably 50 g/l or 150 g/l, or somewhere in between.

Preferably, the composition further comprises a hydrocarbon solvent such as petroleum naphtha. Preferably, the amount of hydrocarbon solvent is between 5% and 30% w/w. More preferably between 10% and 20% w/w.

Preferably, the composition further comprises an alcohol solvent such as ethanol. Preferably, the amount of alcohol solvent is between 1% and 20% w/w. More preferably between 5% and 10% w/w.

The wound to be treated is preferably is selected from the group comprising: chronic wounds, ulcers (such as diabetic ulcers), burns, abrasions, lacerations, punctures and avulsions.

The subject in need of wound healing may be any human or animal, preferably a mammal. More preferably, the subject is a human. In one example, the animal may be a domestic pets (cat, dog etc.), an agricultural animal (pig, cow, sheep, horse, goat etc.), or a non-domestic animal.

Measures of wound healing that may be applied include:
Clinical observations/morphology: raw wound size; percent re-epithelisation; erythema; swelling; and/or granulation;
Histology: epidermal (outer layer of the skin) ulceration (breakage, discontinuity) and acanthosis (thickening); superficial dermal (next deeper layer of skin) congestion/haemorrhage (fluid build-up, bleeding), granulation (formation of new tissue, “proud flesh”) and inflammation; deep dermal granulation, fibrosis (thickening, scarring of connective tissue) and inflammation; subcutal/hyperdermal (bottom layer of the skin) fibrosis and inflammation;
Molecular Analysis: changes in key markers for steps in the healing process such as: chemokines/growth factors, inflammation and migration markers, angiogenesis markers.

A primary advantage of the present invention is expected to be the increased rate at which the wound healing occurs. This enhanced rate of healing is compared to normal wound healing without intervention. The potential for the present invention is widespread, and the topical application of TPP shows promise as an exciting new method of enhancing the rate of wound healing and reducing associated inflammation and scarring.

It is expected that treatment of wounds in accordance with embodiments of the present invention results in improved and more rapid healing of the skin. For example, when used in the treatment of wounds, skin which is treated is expected to heal more quickly and/or completely, compared to when left untreated.

Preferably there is a therapeutically effective amount of TPP in each topical dose of the composition of the present invention.

The amount of composition to be applied will vary. When the composition comprising TPP is administered by spraying a solution of the drug, the total volume in a single dose may be as low as 0.1 ml. When the composition comprising TPP is administered in a gel or cream, the total volume may be as high as 3 ml. Conversely, if there are scattered wounds, the volume applied to each wound may be smaller. The carrier selected, and its manner of application, are preferably chosen in consideration of the needs of the patient and the preferences of the administering physician.

Preferably the composition is administered as a 1 ml aliquot on a bandage that is then applied to the wound as a dressing. The bandage soaked in the composition of the present invention is then covered and temporarily fixed to the skin utilising a covering adhesive moisture vapour permeable film.

The composition of the present invention is preferably administered at about 1 ml/3 cm², or about 0.33 ml/cm². Preferably, between about 2 mg and 100 mg of TPP is administered per cm² of skin. More preferably, between about 5 mg and 50 mg of TPP is administered per cm² of skin.

In one preferred embodiment, the composition comprises a gel which is preferably administered by spreading the gel onto the affected area. In other preferred embodiments, the composition comprises a liquid, which can be administered by spraying or otherwise applying the liquid onto the affected area.

In certain embodiments, the composition of the invention may be provided in a form selected from the group comprising, but not limited to, a liquid, cream or gel, a leave-on preparation, and a wash-off preparation. In one preferred form, the composition is a cream or gel. In another preferred form, the composition is a spray. The composition may or may not contain water. Preferably, the composition does not contain water, i.e. it is non-aqueous.

The TPP could be incorporated into a composition with an additional active moiety that is capable of improving the appearance and/or hydration of the skin.

In addition, the composition of the present invention can be used in conjunction with other topically applied analgesic and/or systemically available agents for the treatment of wounds.

Examples of such analgesic agents include, but are not limited to: morphine, cyclazocine, piperidine, piperazine, pyrrolidine, morphiceptin, meperidine, trifluadom, benzeneacetamine, diacylacetamide, benzomorphan, alkaloids, peptides, phenantrene and pharmaceutically acceptable salts, prodrugs or derivatives thereof. Specific examples of compounds contemplated by as suitable in the present invention include, but are not limited to morphine, heroin, hydromorphone, oxymorphone, levophanol, methadone, meperidine, fentanyl, codeine, hydrocodone, oxycodone, propoxyphene, buprenorphine, butorphanol, pentazocine and nalbuphine. As used in the context of opioid agents herein, “pharmaceutically acceptable salts, prodrugs and derivatives” refers to derivatives of the opioid analgesic compounds that are modified by, e.g., making acid or base salts thereof, or by modifying functional groups present on the compounds in such a way that the modifications are cleaved, either in routine manipulation or in vivo, to produce the analgesically active parent compound. Examples include but are not limited to mineral or organic salts of acidic residues such as amines, alkali or organic salts of acidic residues such as carboxylic acids, acetate, formate, sulfate, tartrate and benzoate derivatives, etc. Suitable opioid analgesic agents, including those specifically mentioned above, are also described in Goodman and Gilman, ibid, chapter 28, pp. 521-555.

In addition, other active agents may be included in the composition of the present invention, e.g., topically-effective anaesthetics such as xylocaine, cocaine, lidocaine, benzocaine, etc., which may provide a more immediate, if less effective in the long run, level of pain relief until the analgesic agent becomes fully effective.

The composition or pharmaceutical composition comprising TPP can be administered to the subject in a range of treatment regimens. For example, the composition or pharmaceutical composition can be administered hourly, three times daily, twice daily, once daily, once every two days, once every three days, once weekly, once every two weeks, once monthly, once every two months, once every six months, and once yearly. The appropriate regimen can be determined by the person skilled in the art based on the nature of the wound to be treated. For example, the pharmaceutical composition may be administered to the wound on days 0, 1, 3, 6, 8, 10 and 13, where day 0 is the day on which the wound was sustained.

### Delivery

The composition is in some embodiments a topical composition. The pharmaceutical composition may be administered topically to body surfaces, and is thus formulated in a form suitable for topical administration. Suitable topical compositions include liposomal beads, gels, ointments, creams, lotions, drops and the like. For topical administration, the TPP is prepared and applied as a solution, suspension, or emulsion in a physiologically acceptable diluent with or without a pharmaceutical carrier. For topical application, admixture of the compound with conventional creams, lotions, or delayed release patches is acceptable. Such a cream or lotion may comprise any agent described herein, and, may be used to treat a dermatological disorder, such as a wound, to enhance healing.

The pharmaceutical composition may also be administered as a topical liquid dosage form such as a solution, suspension, dispersion, emulsion, foam, gel, oil, and the like.

The pharmaceutical composition can be adapted for topical application. In this regard, various topical delivery systems may be appropriate for administering the compositions of the present invention depending up on the preferred treatment regimen. Topical compositions may be produced by dissolving or combining the antibiotics of the present invention in an aqueous or non-aqueous carrier. In general, any liquid, cream, or gel or similar substance that does not appreciably react with the compound or any other of the active ingredients that may be introduced into the composition and which is non-irritating is suitable. Appropriate non-sprayable viscous, semi-solid or solid forms can also be employed that include a carrier compatible with topical application and have dynamic viscosity preferably greater than water.

Suitable compositions are well known to those skilled in the art and include, but are not limited to, solutions, suspensions, emulsions, creams, gels, ointments, powders, liniments, salves, aerosols, transdermal patches, etc, which are, if desired, sterilised or mixed with auxiliary agents, e.g. preservatives, stabilisers, emulsifiers, wetting agents, fragrances, colouring agents, odour controllers, thickeners such as natural gums, etc. Particularly preferred topical compositions include ointments, creams or gels. The topical composition may be non-aqueous.

Ointments generally are prepared using either (1) an oleaginous base, i.e., one consisting of fixed oils or hydrocarbons, such as white petroleum, mineral oil, or (2) an absorbent base, i.e., one consisting of an anhydrous substance or substances which can absorb water, for example anhydrous lanolin. Customarily, following formation of the base, whether oleaginous or absorbent, the active agent is added to an amount affording the desired concentration.

Creams are oil/water emulsions. They consist of an oil phase (internal phase), comprising typically fixed oils, hydrocarbons and the like, waxes, petroleum, mineral oil and the like and an aqueous phase (continuous phase), comprising water and any water-soluble substances, such as added salts. The two phases are stabilised by use of an emulsifying agent, for example, a surface active agent, such as sodium lauryl sulphite, hydrophilic colloids, such as acacia colloidal clays, veegum and the like. Upon formation of the emulsion, the antibiotics can be added in an amount to achieve the desired concentration.

Gels comprise a base selected from an oleaginous base, water, or an emulsion-suspension base. To the base is added a gelling agent that forms a matrix in the base, increasing its viscosity. Examples of gelling agents are hydroxypropyl cellulose, acrylic acid polymers and the like. Customarily, the antibiotics are added to the composition at the desired concentration at a point preceding addition of the gelling agent.

The amount of TPP incorporated into a topical composition is not critical; the concentration should be within a range sufficient to permit ready application of the composition such that an effective amount of the active agent is delivered.

In addition, the pharmaceutical composition may also include other agents. For example, preservatives, co-solvents, surfactants, oils, humectants, emollients, chelating agents, dyestuffs, stabilizers or antioxidants may be employed. Water soluble preservatives that may be employed include, but are not limited to, benzalkonium chloride, chlorobutanol, thimerosal, sodium bisulfate, phenylmercuric acetate, phenylmercuric nitrate, ethyl alcohol, methylparaben, polyvinyl alcohol, benzyl alcohol and phenylethyl alcohol. The surfactant may preferably be polysorbate 80. Other suitable additives include lubricants and slip agents, such as, for example, magnesium stearate, stearic acid, talc and bentonites, substances which promote disintegration, such as starch or cross linked polyvinylpyrrolidone, binders, such as, for example, starch, gelatin or linear polyvinylpyrrolidone, and dry binders, such as microcrystalline cellulose.

Other vehicles that may be used include, but are not limited to, polyvinyl alcohol, povidone, hydroxypropyl methyl cellulose, poloxamers, carboxymethyl cellulose, hydroxyethyl cellulose, purified water, etc. Tonicity adjustors may be included, for example, sodium chloride, potassium chloride, mannitol, glycerin, etc. Antioxidants include, but are not limited to, sodium metabisulfite, sodium thiosulfate, acetylcysteine, butylatedhydroxyanisole, butylated hydroxytoluene, etc. The indications, effective doses, compositions, contraindications, vendors etc, of the active agents in the compositions are available or are known to one skilled in the art.

Electrolytes such as, but not limited to, sodium chloride and potassium chloride may also be included in the composition.

Further, the compositions may contain antimicrobial preservatives. Useful antimicrobial preservatives include methylparaben, propylparaben, benzyl alcohol, phenoxyethanol and hydroxyacetophenone. The microbial preservative is typically employed when the composition is placed in a vial designed for multidose use.

Excipients which may be used are all the physiologically acceptable solid inert substances, either inorganic or organic in nature. Inorganic substances are, for example, sodium chloride, carbonates, such as calcium carbonate, bicarbonates, aluminium oxides, silicic acids, aluminas, precipitated or colloidal silicon dioxide and phosphates. Organic substances are, for example, sugars, cellulose, foodstuffs and feedstuffs, such as milk powder, animal flours, cereal flours and shredded cereals and starches.

Finally, it will be appreciated that the compositions of the present invention may comprise a plurality of active agents as described herein.

The topical composition may be formulated with, but not limited to, pharmaceutically acceptable carriers or diluents, fillers, polymers, glidants, and lubricants.

Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, white paraffin, glycerol, alginates, hyaluronic acid, collagen, perfume oil, fatty acid monoglycerides and diglycerides, pentaerythritol fatty acid esters, hydroxy methylcellulose, and polyvinyl pyrrolidone. The carrier may also comprise any of the substances described in Remington: The Science and Practice of Pharmacy (Gennaro and Gennaro, Eds, 20th edition, Lippincott Williams & Wilkins, 2000); Theory and Practice of Industrial Pharmacy ((Lachman et al., eds., 3.sup.rd edition, Lippincott Williams & Wilkins, 1986); Encyclopedia of Pharmaceutical Technology (Swarbrick and Boylan, eds., 2nd edition, Marcel Dekker, 2002).

The polymers can be chosen from, but not limited to, hydrophilic or hydrophobic polymers such as derivatives of cellulose (for example methylcellulose, hydroxypropyl cellulose, hypromellose, ethylcellulose); polyvinylpirolidone (for example povidone, crospovidone, copovidone); polymethacrylates (for example Eudragit RS, RL); lypophillic components (for example glyceryl monostearate, glyceryl behenate); and various other substances such as for example hydroxypropyl starch, polyethylene oxide, carrageenan and the like. Most commonly, hydrophilic swelling polymers of suitable viscosity such as hypromellose are used, preferably in amounts above 5%, and more preferably above 8%.

Lubricants can be chosen from, but not limited to, stearic acid, magnesium stearate, calcium stearate, aluminium stearate, sodium stearyl fumarate, talc, hydrogenated castor oil, polyethylene glycols and the like.

One of ordinary skill in the art will appreciate that the individual components of the present invention may change depending on the physical and chemical qualities needed for the pharmaceutical compositions in a given process and/or application to which the pharmaceutical compositions will be applied.

### Wound Dressing

The present invention, in some embodiments, provides a wound dressing comprising a pharmaceutical composition comprising triphenyl phosphate or a variant thereof.

A wound dressing includes any covering applied to a wound to promote healing and/or protect the wound from further damage. Wound dressings include, for example, gauze, plastic films, bandage, adhesive bandage, gels, alginates, composite materials, hydrocolloids, semipermeable films and hydrogels.

Preferably, the wound dressing may be a sterile gauze bandage, which has been saturated with the pharmaceutical composition comprising triphenyl phosphate or a variant thereof. The wound dressing can be applied directly to the wound to enhance wound healing.

### Kits

The present invention provides a kit comprising
i) a pharmaceutical composition for enhancing wound healing in a subject in need thereof, said composition comprising:
   a) triphenyl phosphate.

In a further embodiment, the kit also comprises instructions for its use.

In one aspect, the present invention provides a unit dosage form comprising:
i) a pharmaceutical composition for enhancing wound healing in a subject in need thereof, said composition comprising triphenyl phosphate.

Preferably, the amount of triphenyl phosphate is between 10 and 200 g/l; more preferably 50 g/l or 150 g/l, or somewhere in between.

Preferably the composition further comprises a hydrocarbon solvent such as petroleum naphtha. Preferably, the amount of hydrocarbon solvent is between 5% and 30% w/w, more preferably between 10% and 20% w/w.

Preferably the composition further comprises an alcohol solvent such as ethanol. Preferably, the amount of alcohol solvent is between 1% and 20% w/w, more preferably between 5% and 10% w/w.

The contents of the kit can be lyophilized and the kit can additionally contain a suitable solvent for reconstitution of the lyophilized components. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale for human or animal administration.

When the components of the kit are provided in one or more liquid solutions, the liquid solution can be an aqueous solution or a non-aqueous solution. Preferably the solution is sterile. For in vivo use, the TPP may be formulated into a pharmaceutically acceptable topically applicable composition in an applicator. In this case the container means may itself be a syringe, pipette, eye dropper, or other such like apparatus, from which the formulation may be applied to an affected area of the animal, or even applied to and mixed with the other components of the kit.

The components of the kit may also be provided in dried or lyophilized forms. When reagents or components are provided as a dried form, reconstitution generally is by the addition of a suitable solvent. It is envisioned that the solvent also may be provided in another container means. Irrespective of the number or type of containers, the kits of the invention also may comprise, or be packaged with, an instrument for assisting with the administration or placement of the ultimate composition topically on the body of an animal. Such an instrument may be a syringe, pipette, measured spoon, eye dropper or any such medically approved delivery vehicle.

### General

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (eg. Size, displacement and field strength etc). A range of values will be understood to include all values within the range, including the values defining the range, and values adjacent to the range which lead to the same or substantially the same outcome as the values immediately adjacent to that value which defines the boundary to the range. Accordingly, unless indicated to the contrary, the numerical parameters set forth in the specification and claims are approximations that may vary depending upon the desired properties sought to be obtained by the present invention. Hence “about 80%” means “about 80%” and also “80%”. At the very least, each numerical parameter should be construed in light of the number of significant digits and ordinary rounding approaches.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs. The term “active agent” may mean one active agent, or may encompass two or more active agents.

The following examples serve to more fully describe the manner of using the above-described invention, as well as to set forth the best modes contemplated for carrying out various aspects of the invention. It is understood that these methods in no way serve to limit the true scope of this invention, but rather are presented for illustrative purposes.

### EXAMPLES

Further features of the present invention are more fully described in the following non-limiting Examples. This description is included solely for the purposes of exemplifying the present invention. It should not be understood as a restriction on the broad description of the invention as set out above.

### Example 1

### Pig-Model Wound Healing

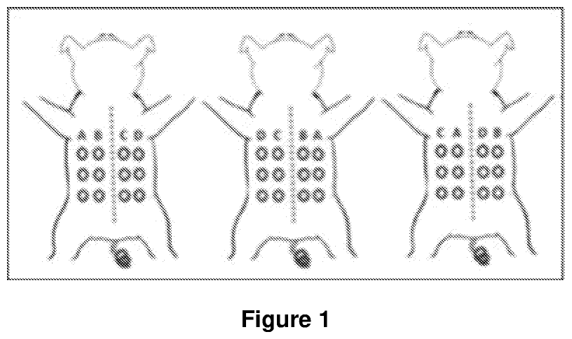

Twelve female pathogen free Domestic Yorkshire-cross pigs were used. Test samples were randomly distributed and replicated among three animals as shown in FIG. 1.

TABLE 1
Compositions tested

| Code | Sample | Active |
|---|---|---|
| A | Carrier Control | Carrier only (37% w/w 95% ethanol; |
| | | 63% w/w petroleum naphtha |
| | | Rescol 150) |
| B | Active, Conc 1 | 150 g/l TPP in Carrier |
| C | Active, Conc 2 | 50 g/l TPP in Carrier |
| D | Master control | 0.9% w/w saline |

Wounds were initially 2 cm in diameter and about 0.5 cm deep. Test and Control samples were applied on Days 0, 1, 3, 6, 8 and 10.

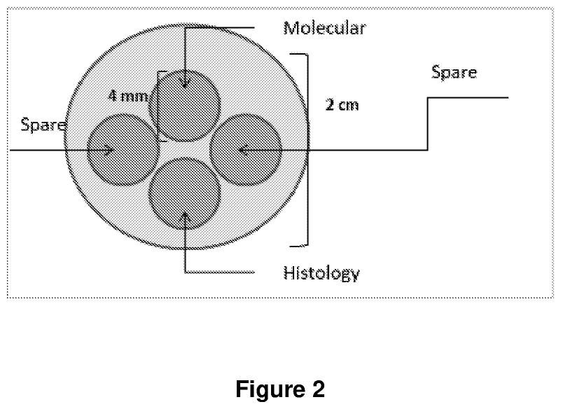

Wounds were sampled for analysis as in FIG. 2. Sampling was undertaken on Days 6, 8, 10 and 13. Samples were applied under general anaesthesia and the wounds were photographed on each sampling day. Each wound was covered with a sterile gauze bandage saturated with 1 ml of test composition then covered with a transparent adhesive moisture vapour permeable film such as Opsite®.

The overall morphology (appearance) of the wound was clinically assessed at days 0, 1, 3, 6, 8, 10 and 13. At days 6, 8, 10 and 13 wounds were sampled as shown for histological and molecular analysis.

### Wound Morphology

Raw wound size was determined on Day 0, 1, 3, 6, 8, 10 and 13. At these time points, each wound was also evaluated for redness (Erythema), swelling and the production of new tissue over the wound (Granulation).

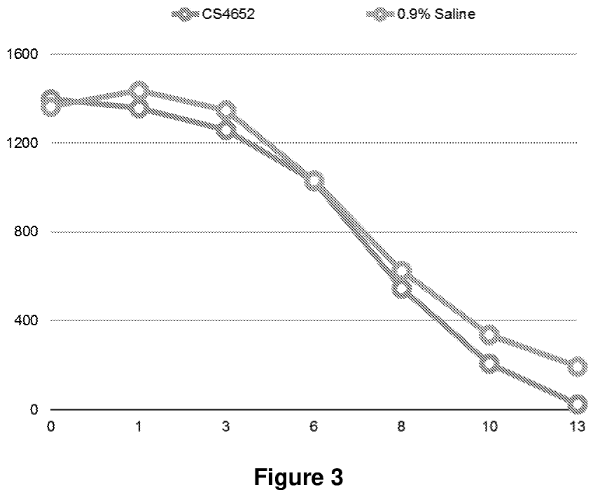

CS4652 treated wounds exhibited a moderate but consistent acceleration of wound healing as measured by raw wound size particularly for the later stages (Days 8, 10 and 13) of the experiment. A plot of wound size over time of a typical animal treated with 50 g/L CS4652 for the entire time course of the experiment is shown in FIG. 3.

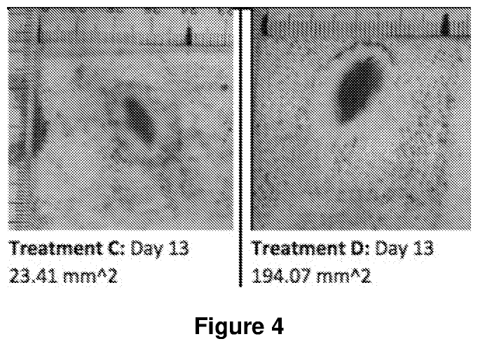

Animal #6 raw wound sizes (mm²) were derived from digitised images utilising ImageJ and plotted vs time (Days). On Day 13, the 50 g/L CS4652 granulating wound bed area was 15% of the control 0.9% saline granulating wound bed area (29 mm² vs 195 mm²); see FIG. 4.

It is important to note that this is an outbred animal model system with more variability than standard inbred animal models and thus more closely represents what would be expected from human trial studies.

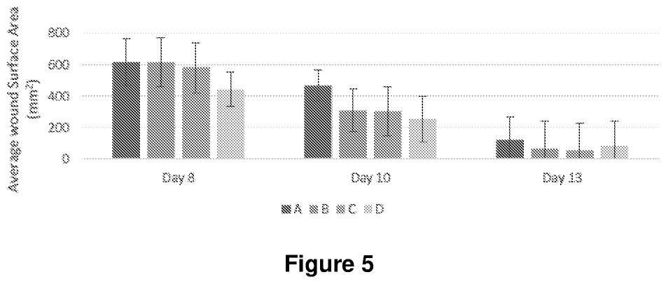

The average wound size for all animals over the later stages (Days 8, 10 and 13) is shown in FIG. 5.

Erythema and Swelling scores (0=Normal, 1=slight, 2=moderate, 3=severe, 4=very severe) were low, particularly at the later stages of the experiment. The 9 g/L w/w CS4652 formulation values were very close to the 0.9% Saline master control (<0.4 average).

There appears to be a slight lag in the acceleration of CS4652 wound healing as the average wound size is may be slightly larger than the Saline Control until about day 8 and then the CS4652 wound beds resolve themselves considerably faster than the Saline Controls.

These data indicate that the CS4652 formulation moderately accelerates even efficient wound healing under normal conditions.

Clinical scores for granulation, epithelialisation, erythema and swelling indicate that the CS4652 does not have a deleterious effect on the quality of wound healing it just speeds up the process as measured by wound bed area.

### Histological Analysis

Wound samples were taken and sectioned for histopathological analysis at days 6, 8, 10 and 13.

Slides were analysed blindly then grouped by treatment. The epidermis (outer skin layer) was scored for Ulceration (breakage, discontinuity) and Acanthosis (thickening). The superficial dermis (next deeper layer of skin) was scored for Congestion/Haemorrhage (fluid build-up, bleeding), Granulation (formation of new tissue, “proud flesh”) and Inflammation. The deep dermis was scored for Granulation, Fibrosis (thickening, scarring of connective tissue) and Inflammation. Finally the subcutis/hyperdermis (bottom layer of the skin) was scored for Fibrosis and Inflammation.

By Day 8, the 9 g/L CS54652 and the 0.9% Saline master control samples exhibited the most advanced healing. By Day 10, the healing process of the Carrier Control and 150 g/uL CS5462 formulation samples were beginning to catch up. By Day 13, epithelial healing in the 150 g/L formulation has caught up to the 50 g/L and the 0.9% Saline master control. Of the three samples, the 0.9% Saline solution had slightly less inflammation and a slightly better resolution of the fibrous reaction though the wound bed is shrinking more rapidly in the CS4652 treatment samples. The differences, however, between Day 13 CS4652 and master control Day 13 0.9% saline were subtle and demonstrate that the CS4652 formulation moderately accelerates normal wound healing without effecting the production of appropriately regenerated normal skin.

RNA was isolated from wound samples at Days 6, 8, 10 and 13. The RNA was reverse transcribed into cDNA utilising Omniscript Reverse Transcriptase (Qiagen). The relative amount of the following transcripts, previously shown to be relevant for wound healing analysis were determined by quantifiable polymerase chain reaction.

### Gene Products Relevant to Wound Healing

Gene products tested:
18S Ribosomal RNA
Actin, Beta (ACTB)
Glyceraldehyde-3-Phosphate Dehydrogenase (GAPDH)
Prostaglandin-Endoperoxide Synthase 2 (PTGS2)
Connective Tissue Growth Factor (CTGF)
Intercellular Adhesion Molecule 1 (ICAM1)
Insulin-Like Growth Factor 1 (IGF1)
Interleukin 1-Beta (IL1B)
Interleukin 6 (IL6)
Interleukin 8 (IL8)
Nitric Oxide Synthase 2A (NOS2A)
Matrix Metalloproteinase 1 (MMP1)
Matrix Metalloproteinase 2 (MMP2)
Matrix Metalloproteinase 9 (MMP9)
Transforming Growth Factor, Beta-1 (TGFB1)
Tumor Necrosis Factor (TNF)
Vascular Endothelial Growth Factor A (VEGFA)

### Molecular Protocol Overview:

Skin samples from the experimental study were obtained from separate groups of pigs at Day 6, 8, 10 and 13 post-wounding, and were obtained from CCR frozen on dry ice after being removed from the pigs, weighed, and immediately frozen in liquid nitrogen and maintained at −80° C. On arrival, the samples remained frozen and an excess of dry ice was still evident. The samples were immediately placed in a −80° C. freezer until processing.

All samples (uninjured skin, carrier (A); 50 g/L CS54652 low dose intervention (B); 150 g/L CS54652 high dose intervention (C); and saline (D)) were subjected to powdering with a Braun Microdismembrator. One ml of Trizol was immediately added to the powdered samples in preparation for total RNA isolation. Total RNA was processed and quantified by established methods. The protein layers from the Trizol extraction were immediately stored at 4° C. for any future use.

Aliquots (1 microgram) of total RNA from each sample was reverse transcribed using an Omniscript RT kit from Qiagen and subsequently used for qPCR analysis for a subset of relevant genes (housekeeping, inflammatory mediators, matrix metalloproteinases, growth factors). The primer sets used for the qPCR analysis were porcine specific and validated. The SybrGreen method for qPCR was performed with a BioRad iCycler. Samples from each day of isolation+control skin (normal from a separate group of pigs) were assessed by qPCR on the same plate.

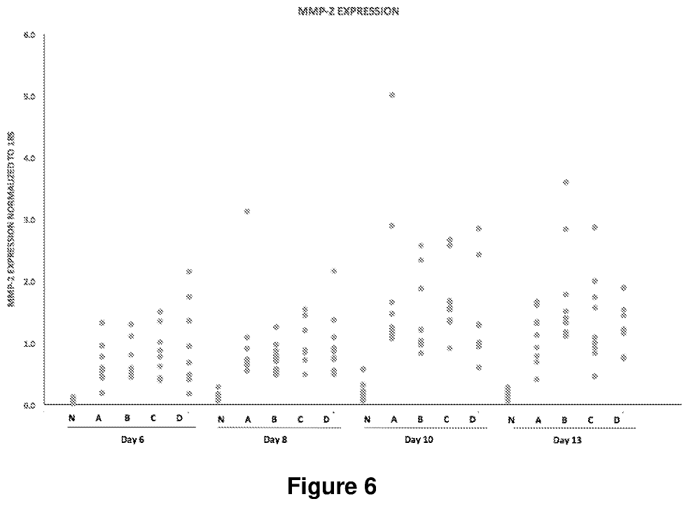

The three gene products at the top of the table represent “Housekeeping” genes that are present in reasonably stable amounts in most cell types and provide a baseline for comparing relative amounts of genes that are up regulated and down regulated during the wound healing process. Typical results exemplified by the Matrix Metalloproteinase 2 gene that regulates cell migration during wound healing is shown in FIG. 6.

In all cases, no aberrant gene expression profiles outside experimental error were detected.

The study demonstrates that the CS4652 formulation moderately enhances normal wound healing as measured by the reduction in wound bed area over time without effecting the efficient regeneration of normal skin tissue. The formulation exhibits no overt skin toxicity in an animal model that is considered to be the closest animal model system to human skin available.

Numerous variations and modifications of the above-described modes of carrying out the various embodiments of this invention will be apparent to those skilled in the art, based on the above teachings related to the disclosed invention, without departing from the basic inventive concepts. The above embodiments of the invention are merely exemplary and should not be construed to be in any way limiting and all such variations and modifications are to be considered within the scope of the present invention, the nature of which is to be determined from the foregoing description.

### REFERENCES

Atiyeh, B. S., Dibo, S. A. and Hayek, S. N. (2009), Wound cleansing, topical antiseptics and wound healing. International Wound Journal, 6: 420-430
Ashcroft, G. S., & Mills, S. J. (2002). Androgen receptor-mediated inhibition of cutaneous wound healing. J Clin Invest, 110(5), 615-624.
Bardaa, S., Chabchoub, N., Jridi, M., Moalla, D., Mseddi, M., Rebai, T., & Sahnoun, Z. (2016). The effect of natural extracts on laser burn wound healing. J Surg Res, 201(2), 464-472.
Braun, S., Hanselmann, C., Gassmann, M. G., auf dem Keller, U., Born-Berclaz, C., Chan, K., Werner, S. (2002). Nrf2 transcription factor, a novel target of keratinocyte growth factor action which regulates gene expression and inflammation in the healing skin wound. Mol Cell Biol, 22(15), 5492-5505.
Bucala, R., Spiegel, L. A., Chesney, J., Hogan, M., & Cerami, A. (1994). Circulating fibrocytes define a new leukocyte subpopulation that mediates tissue repair. Mol Med, 1(1), 71-81.
Campbell, L., Emmerson, E., Davies, F., Gilliver, S. C., Krust, A., Chambon, P., and Hardman, M. J. (2010). Estrogen promotes cutaneous wound healing via estrogen receptor beta independent of its antiinflammatory activities. J Exp Med, 207(9), 1825-1833.
Enoch, S., Price, P. (2004) Cellular, molecular and biochemical differences in the pathophysiology of healing between acute wounds, chronic wounds and wounds in the aged.
Goyal, A., Sharma, V., Upadhyay, N., Gill, S., & Sihag, M. (2014). Flax and flaxseed oil: an ancient medicine & modern functional food. J Food Sci Technol, 51(9), 1633-1653.
Harding, K. G., Morris, H. L., Patel, G. K. (2002) Science, medicine and the future: healing chronic wounds. Bmj 324, 160-3.
Han, G. and Ceilley, R. (2017) Chronic Wound Healing: A Review of Current Management and Treatments. Adv Ther in press.
Hudson, G., Zamponi, A., Alston, L., Chang, T., & Hirota, S. A. (2016). Activation Of The Constitutive Androstane Receptor Enhances Wound Healing—A New Link Between Xenobiotic Sensing And Intestinal Mucosal Homeostasis?. The FASEB Journal, 30(1 Supplement), 1250-3.
Julovi, S. M., Xue, M., Dervish, S., Sambrook, P. N., March, L., & Jackson, C. J. (2011). Protease activated receptor-2 mediates activated protein C-induced cutaneous wound healing via inhibition of p38. Am J Pathol, 179(5), 2233-2242.
Lai, J. J., Lai, K. P., Chuang, K. H., Chang, P., Yu, I. C., Lin, W. J., & Chang, C. (2009). Monocyte/macrophage androgen receptor suppresses cutaneous wound healing in mice by enhancing local TNF-alpha expression. J Clin Invest, 119(12), 3739-3751.
Lewinska, A., Zebrowski, J., Duda, M., Gorka, A., & Wnuk, M. (2015b). Fatty Acid Profile and Biological Activities of Linseed and Rapeseed Oils. Molecules, 20(12), 22872-22880.
Mann, A., Breuhahn, K., Schirmacher, P., Blessing, M. (2001) Keratinocyte-derived granulocyte-macrophage colony stimulating factor accelerates wound healing: Stimulation of keratinocyte proliferation, granulation tissue formation, and vascularization. J Invest Dermatol 117, 1382-90.
Martin, P. (1997). Wound healing—aiming for perfect skin regeneration. Science 276, 75-81.
Moore, K. (2001) The scientific basis of wound healing. Adv Tissue Banking 5, 379-397.
Sedlarik, K. M. (2003) The process of wound healing. Wound Forum Update
Süntar, I., Tumen, I., Ustün, O., Keleş, H., & Akkol, E. K. (2012). Appraisal on the wound healing and anti-inflammatory activities of the essential oils obtained from the cones and needles of Pinus species by in vivo and in vitro experimental models. J Ethnopharmacol, 139(2), 533-540.
Terc, J., Hansen, A., Alston, L., & Hirota, S. A. (2014). Pregnane X receptor agonists enhance intestinal epithelial wound healing and repair of the intestinal barrier following the induction of experimental colitis. Eur J Pharm Sci, 55, 12-19.
Werner, S. and Grose, R. (2003). Regulation of wound healing by growth factors and cytokines. Physiol Rev. 83, 835-870.
Zhao, R. et al. (2016) Inflammation in Chronic Wounds. Int J Mol Sci, v. 17, n. 12.

1-4. (canceled)

5. A pharmaceutical composition comprising:
a) triphenyl phosphate (TPP) or variant thereof; and
b) one or more pharmaceutically acceptable carriers, excipients or diluents thereof.

6. The pharmaceutical composition of claim 5, wherein the pharmaceutical composition is adapted for topical administration to a subject in need thereof.

7. The pharmaceutical composition of claim 5, wherein the concentration of TPP is between 10 g/l and 200 g/l.

8. The pharmaceutical composition of claim 5, wherein the composition comprises a hydrocarbon solvent.

9. The pharmaceutical composition of claim 8, wherein the hydrocarbon solvent is petroleum naphtha.

10. The pharmaceutical composition of claim 8, wherein the concentration of hydrocarbon solvent is between 5% and 30% w/w.

11. The pharmaceutical composition of claim 5, wherein the composition further comprises an alcohol solvent.

12. The pharmaceutical composition of claim 11, wherein the alcohol solvent is ethanol.

13. The pharmaceutical composition of claim 11, wherein the amount of alcohol solvent is between 1% and 20% w/w.

14. The pharmaceutical composition of claim 5, wherein the pharmaceutical composition is a liquid, ointment, spray, cream or gel.

15. A method of enhancing wound healing in a subject in need thereof, comprising administering to the wound of the subject, a therapeutically effective amount of the pharmaceutical composition of claim 5.

16. A wound dressing comprising the pharmaceutical composition of claim 5.

17-18. (canceled)

19. A kit for enhancing wound healing in a subject comprising the pharmaceutical composition of claim 5.

20. (canceled)

21. The pharmaceutical composition of claim 5, wherein the concentration of TPP is between 50 g/l and 150 g/1.

22. The pharmaceutical composition of claim 8, wherein the concentration of hydrocarbon solvent is between 10% and 20% w/w.

23. The pharmaceutical composition of claim 11, wherein the amount of alcohol solvent is between 5% and 10% w/w.
